(19) United States
(12) Patent Application Publication
(10) Pub. No.: US 2004/0138821 A1
(43) Pub. Date: Jul. 15, 2004

(54) System, method, and computer software product for analysis and display of genotyping, annotation, and related information

(75) Inventors: Richard Chiles, Castro Valley, CA (US); Muniyappa Prakash, San Jose, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, Inc. a Corporation Organized under the laws of Delaware

(21) Appl. No.: 10/657,481

(22) Filed: Sep. 8, 2003

Related U.S. Application Data
(60) Provisional application No. 60/408,848, filed on Sep. 6, 2002.
(60) Provisional application No. 60/423,073, filed on Nov. 1, 2002.

(51) Int. Cl. G01N 33/48; G06G 7/48

(52) U.S. Cl. 702/19; 703/11

(57) ABSTRACT

A method for displaying genotype information associated with probe array experiments is described that includes the acts of receiving sets of emission intensity data, wherein each set of emission intensity data includes emission intensity values each associated with a probe disposed upon a probe array; generating genotype calls, wherein each of the genotype calls is based, at least in part, upon the emission intensity values; assembling the genotype calls into one or more genotype data sets; and displaying each of the genotype data sets in one or more panes of a graphical user interface.

### RELATED APPLICATIONS

The present application claims priority to U.S. Provisional Patent Application Serial Nos. 60/408,848, titled “System, Method, and Computer Software Product for Determination and Comparison of Biological Sequence Composition”, filed Sep. 6, 2002; and 60/423,073, titled “Computer Software for Analyzing Genotype Data”, filed Nov. 1, 2002, each of which is hereby incorporated by reference herein in its entirety for all purposes. The present application is also related to U.S. patent application Ser. No. 10/219,503, titled “System, Method, and Computer Software for Genotyping Analysis and Identification of Allelic Imbalance”, filed Aug. 15, 2002, which is hereby incorporated by reference herein in its entirety for all purposes.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### BACKGROUND

1. Field of the Invention

The present invention relates to the field of bioinformatics. In particular, the present invention relates to computer systems, methods, and products for the storage and presentation of data resulting from the analysis of microarrays of biological materials.

2. Related Art

Research in molecular biology, biochemistry, and many related health fields increasingly requires organization and analysis of complex data generated by new experimental techniques. The rapidly evolving field of bioinformatics addresses these tasks. See, e.g., H. Rashidi and K. Buehler, Bioinformatics Basics: Applications in Biological Science and Medicine (CRC Press, London, 2000); Bioinformatics: A Practical Guide to the Analysis of Gene and Proteins (B. F. Ouelette and A. D. Bzevanis, eds., Wiley & Sons, Inc.; 2d ed., 2001), both of which are hereby incorporated herein by reference in their entireties. Broadly, one area of bioinformatics applies computational techniques to large genomic databases, often distributed over and accessed through networks such as the Internet, for the purpose of illuminating relationships among gene structure and/or location, protein function, and metabolic processes.

The expanding use of microarray technology is one of the forces driving the development of bioinformatics. Spotted arrays, such as those made using the Affymetrix® 417™ or 427™ Arrayer from Affymetrix, Inc. of Santa Clara, Calif., are used to generate information about biological systems. Also, synthesized probe arrays, such as Affymetrix® GeneChip® arrays, have been widely used to generate unprecedented amounts of information about biological systems. For example, the GeneChip® Human Genome U133 Set (HG-U133A and HG-U133B) is made up of two microarrays containing over 1,000,000 unique oligonucleotide features covering more than 39,000 transcript variants that represent more than 33,000 human genes. Experimenters can quickly design follow-on experiments with respect to genes, EST's, or other biological materials of interest by, for example, producing in their own laboratories microscope slides containing dense arrays of probes using the Affymetrix® 417™ or 427™ Arrayer, or other spotting device.

Analysis of data from experiments with synthesized and/or spotted probe arrays may lead to the development of new drugs and new diagnostic tools. In some applications, this analysis begins with the capture of fluorescent signals indicating hybridization of labeled target samples with probes on synthesized or spotted probe arrays. The devices used to capture these signals often are referred to as scanners, an example of which is the Affymetrix® 428™ Scanner.

There is a great demand in the art for methods for organizing, accessing and analyzing the vast amount of information collected by scanning microarrays. Computer-based systems and methods have been developed to assist a user to obtain, analyze, and visualize the vast amounts of information generated by the scanners. These commercial and academic software applications typically provide such information as intensities of hybridization reactions or comparisons of hybridization reactions. This information may be displayed to a user in graphical form. In particular, data representing detected emissions conventionally are stored in a memory device of a computer for processing. The processed images may be presented to a user on a video monitor or other device, and/or operated upon by various data processing products or systems.

In particular, microarrays and associated instrumentation and computer systems have been developed for rapid and large-scale collection of data, including the expression of genes or expressed sequence tags (EST's) in tissue samples, as well as sequence information from one or more samples of DNA such as, for instance, what are referred to as Single Nucleotide Polymorphisms hereafter referred to as SNP's. The data may be used, among other things, to study genetic characteristics and to detect mutations relevant to genetic and other diseases or conditions. More specifically, the data gained through microarray experiments is valuable to researchers because, among other reasons, many disease states can potentially be characterized by differences in the expression levels of various genes, either through changes in the copy number of the genetic DNA or through changes in levels of transcription (e.g., through control of initiation, provision of RNA precursors, or RNA processing) of particular genes. Alternatively, the presence of a particular SNP or multiple SNP's may be associated with a specific disease or condition that may alter the expression or function of one or more protein products. Thus, for example, researchers use microarrays to answer questions such as: Which genes are expressed in cells of a malignant tumor but not expressed in either healthy tissue or tissue treated according to a particular regime? Which genes or EST's are expressed in particular organs but not in others? Which genes or EST's are expressed in particular species but not in others? How does the environment, drugs, or other factors influence gene expression? Which SNP's are present that indicate a predisposition to some disease or condition? Data collection is only an initial step, however, in answering these and other questions. Researchers are increasingly challenged to extract biologically meaningful information from the vast amounts of data generated by microarray technologies, and to design follow-on experiments. A need exists to provide researchers with improved tools and information to perform these tasks.

### SUMMARY OF THE INVENTION

Systems, methods, and products to address these and other needs are described herein with respect to illustrative, non-limiting, implementations. Various alternatives, modifications and equivalents are possible. For example, certain systems, methods, and computer software products are described herein using exemplary implementations for analyzing data from arrays of biological materials produced by the Affymetrix® 417™ or 427™ Arrayer. Other illustrative implementations are referred to in relation to data from Affymetrix® GeneChip® probe arrays. However, these systems, methods, and products may be applied with respect to many other types of probe arrays and, more generally, with respect to numerous parallel biological assays produced in accordance with other conventional technologies and/or produced in accordance with techniques that may be developed in the future. For example, the systems, methods, and products described herein may be applied to parallel assays of nucleic acids, PCR products generated from cDNA clones, proteins, antibodies, or many other biological materials. These materials may be disposed on slides (as typically used for spotted arrays), on substrates employed for GeneChip® arrays, or on beads, optical fibers, or other substrates or media, which may include polymeric coatings or other layers on top of slides or other substrates. Moreover, the probes need not be immobilized in or on a substrate, and, if immobilized, need not be disposed in regular patterns or arrays. For convenience, the term “probe array” will generally be used broadly hereafter to refer to all of these types of arrays and parallel biological assays.

A method for displaying genotype information associated with probe array experiments is described that includes the acts of receiving sets of emission intensity data, wherein each set of emission intensity data includes emission intensity values each associated with a probe disposed upon a probe array; generating genotype calls, wherein each of the genotype calls is based, at least in part, upon the emission intensity values; assembling the genotype calls into one or more genotype data sets; and displaying each of the genotype data sets in one or more panes of a graphical user interface.

In some embodiments, each of the emission intensity values corresponds to detected emissions from a scanned probe array. Also, the probe includes a genotyping probe such as a sequencing probe or a SNP probe. In some implementations, genotype call includes an A, G, C, T, or (n) call that refers to an identified nucleotide associated with a sequencing call or a SNP call.

In the same or alternative embodiments, the graphical user interface includes one or more panes enabled to display information in a tabular or graphical format. In some implementations, graphical format may include a representation of relative SNP call quality, genotype calls associated with a representation of a sequence, or a representation of probe intensity.

Some embodiments may also further include the acts of retrieving annotation information in response to a user selection of one or more of the displayed genotype calls; and displaying the annotation information in one or more panes of the graphical user interface.

A system for displaying genotype information associated with probe array experiments is described that includes a sequence data manager that receives sets of emission intensity data, wherein each set of emission intensity data includes emission intensity values each associated with a probe disposed upon a probe array; a genotype call generator that generates genotype calls, wherein each of the genotype calls is based, at least in part, upon one or more of the emission intensity values; a data assembler that assembles the genotype calls into one or more genotype data sets; and an output manager that displays each of the one or more genotype data sets in one or more panes of a graphical user interface.

A computer system for displaying genotype information associated with probe array experiments is described that includes a user computer having system memory with executable code, wherein the executable code performs the acts of receiving sets of emission intensity data, wherein each set of emission intensity data includes emission intensity values each associated with a probe disposed upon a probe array; generating genotype calls, wherein each of the genotype calls is based, at least in part, upon one or more of the emission intensity values; assembling the genotype calls into one or more genotype data sets; and displaying each of the one or more genotype data sets in one or more panes of a graphical user interface.

The above implementations are not necessarily inclusive or exclusive of each other and may be combined in any manner that is non-conflicting and otherwise possible, whether they be presented in association with a same, or a different, aspect or implementation. The description of one implementation is not intended to be limiting with respect to other implementations. Also, any one or more function, step, operation, or technique described elsewhere in this specification may, in alternative implementations, be combined with any one or more function, step, operation, or technique described in the summary. Thus, the above implementations are illustrative rather than limiting.

### BRIEF DESCRIPTION OF THE DRAWINGS

In the drawings, like reference numerals indicate like structures or method steps and the leftmost digit of a reference numeral indicates the number of the figure in which the referenced element first appears (for example, the element 120 appears first in FIG. 1). In functional block diagrams, rectangles generally indicate functional elements, parallelograms generally indicate data, and rectangles with a pair of double borders generally indicate predefined functional elements. These conventions, however, are intended to be typical or illustrative, rather than limiting.

FIG. 1 is a functional block diagram of one embodiment of a computer system including illustrative embodiments of probe array analysis executables and display/output devices including graphical user interfaces;

FIG. 2 is a functional block diagram of one embodiment of the computer system of FIG. 1 connected to a user-side Internet client and database server via a network for communication over the Internet;

FIG. 3 is a functional block diagram of one embodiment of the probe array analysis executables of FIG. 1 including illustrative embodiments of a sequence data manager and an output manager;

FIG. 4A is graphical representation of one embodiment of an interactive graphical user interface displaying the results of one or more microarray experiments in a tabular format;

FIG. 4B is graphical representation of one embodiment of an interactive graphical user interface displaying a plurality of panes each providing sequence information at varying degrees of resolution;

FIG. 5 is graphical representation of one embodiment of an interactive graphical user interface displaying probe intensity information; and

FIG. 6 is graphical representation of one embodiment of an interactive graphical user interface displaying single nucleotide polymorphism information.

### DETAILED DESCRIPTION

User Computer 100: User computer 100 may be a computing device specially designed and configured to support and execute some or all of the functions of probe array applications 199, described below. Computer 100 also may be any of a variety of types of general-purpose computers such as a personal computer, network server, workstation, or other computer platform now or later developed. Computer 100 typically includes known components such as a processor 105, an operating system 110, a graphical user interface (GUI) controller 115, a system memory 120, memory storage devices 125, and input-output controllers 130. It will be understood by those skilled in the relevant art that there are many possible configurations of the components of computer 100 and that some components that may typically be included in computer 100 are not shown, such as cache memory, a data backup unit, and many other devices. Processor 105 may be a commercially available processor such as a Pentium® processor made by Intel Corporation, a SPARC® processor made by Sun Microsystems, or it may be one of other processors that are or will become available. Processor 105 executes operating system 110, which may be, for example, a Windows®-type operating system (such as Windows NT® 4.0 with SP6a) from the Microsoft Corporation; a Unix® or Linux-type operating system available from many vendors; another or a future operating system; or some combination thereof. Operating system 110 interfaces with firmware and hardware in a well-known manner, and facilitates processor 105 in coordinating and executing the functions of various computer programs that may be written in a variety of programming languages. Operating system 110, typically in cooperation with processor 105, coordinates and executes functions of the other components of computer 100. Operating system 110 also provides scheduling, input-output control, file and data management, memory management, and communication control and related services, all in accordance with known techniques.

System memory 120 may be any of a variety of known or future memory storage devices. Examples include any commonly available random access memory (RAM), magnetic medium such as a resident hard disk or tape, an optical medium such as a read and write compact disc, or other memory storage device. Memory storage device 125 may be any of a variety of known or future devices, including a compact disk drive, a tape drive, a removable hard disk drive, or a diskette drive. Such types of memory storage device 125 typically read from, and/or write to, a program storage medium (not shown) such as, respectively, a compact disk, magnetic tape, removable hard disk, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory 120 and/or the program storage device used in conjunction with memory storage device 125.

In some embodiments, a computer program product is described comprising a computer usable medium having control logic (computer software program, including program code) stored therein. The control logic, when executed by processor 105, causes processor 105 to perform functions described herein. In other embodiments, some functions are implemented primarily in hardware using, for example, a hardware state machine. Implementation of the hardware state machine so as to perform the functions described herein will be apparent to those skilled in the relevant arts.

Input-output controllers 130 could include any of a variety of known devices for accepting and processing information from a user, whether a human or a machine, whether local or remote. Such devices include, for example, modem cards, network interface cards, sound cards, or other types of controllers for any of a variety of known input devices 102. Output controllers of input-output controllers 130 could include controllers for any of a variety of known display devices 180 for presenting information to a user, whether a human or a machine, whether local or remote. If one of display devices 180 provides visual information, this information typically may be logically and/or physically organized as an array of picture elements, sometimes referred to as pixels. Graphical user interface (GUI) controller 115 may comprise any of a variety of known or future software programs for providing graphical input and output interfaces between computer 100 and user 175, and for processing user inputs. In the illustrated embodiment, the functional elements of computer 100 communicate with each other via system bus 104. Some of these communications may be accomplished in alternative embodiments using network or other types of remote communications.

As will be evident to those skilled in the relevant art, applications 199, if implemented in software, may be loaded into system memory 120 and/or memory storage device 125 through one of input devices 102. All or portions of applications 199 may also reside in a read-only memory or similar device of memory storage device 125, such devices not requiring that applications 199 first be loaded through input devices 102. It will be understood by those skilled in the relevant art that applications 199, or portions of it, may be loaded by processor 105 in a known manner into system memory 120, or cache memory (not shown), or both, as advantageous for execution.

Scanner 150: Scanner 150 of this example may provide pixel intensity data that could be further processed into an image of hybridized probe-target pairs by detecting fluorescent, radioactive, or other emissions; by detecting transmitted, reflected, or scattered radiation; by detecting electromagnetic properties or characteristics; or by other techniques. These processes or techniques may generally and collectively be referred to hereafter for convenience simply as involving the detection of “emissions.” Various detection schemes are employed depending on the type of emissions and other factors. A typical scheme employs optical and other elements to provide excitation light and to selectively collect the emissions. Also generally included are various light-detector systems employing photodiodes, charge-coupled devices, photomultiplier tubes, or similar devices to register the collected emissions. For example, a scanning system for use with a fluorescent label is described in U.S. Pat. No. 5,143,854, which is hereby incorporated by reference herein in its entirety for all purpose. Illustrative scanners or scanning systems that, in various implementations, may include scanner 150 are described in U.S. Pat. Nos. 5,143,854, 5,578,832, 5,631,734, 5,834,758, 5,936,324, 5,981,956, 6,025,601, 6,141,096, 6,185,030, 6,201,639, 6,218,803, and 6,252,236; in PCT Application PCT/US99/06097 (published as WO99/47964); in U.S. patent application Ser. Nos. 10/063,284, 09/683,216, 09/683,217, 09/683,219, 09/681,819, and 09/383,986; and in U.S. Provisional Patent Applications Serial Nos. 60/364,731, and 60/286,578, each of which is hereby incorporated herein by reference in its entirety for all purposes.

Scanner 150 of this non-limiting example provides data representing the intensities (and possibly other characteristics, such as color) of the detected emissions, as well as the locations on the substrate where the emissions were detected. The data typically are stored in a memory device, such as system memory 120 of user computer 150, in the form of a data file. One type of data file, such as image data 176 that could for example be in the form of a “*.cel” file generated by Microarray Suite software available from Affymetrix, Inc., typically includes intensity and location information corresponding to elemental sub-areas of the scanned substrate. In the illustrated example, data 176 could be received by computer 100 where a *.cel file could be generated or the *.cel file could be generated by scanner 150. The term “elemental” in this context means that the intensities, and/or other characteristics, of the emissions from this area each are represented by a single value. When displayed as an image for viewing or processing, elemental picture elements, or pixels, often represent this information. Thus, for example, a pixel may have a single value representing the intensity of the elemental sub-area of the substrate from which the emissions were scanned. The pixel may also have another value representing another characteristic, such as color. For instance, a scanned elemental sub-area in which high-intensity emissions were detected may be represented by a pixel having high luminance (hereafter, a “bright” pixel), and low-intensity emissions may be represented by a pixel of low luminance (a “dim” pixel). Alternatively, the chromatic value of a pixel may be made to represent the intensity, color, or other characteristic of the detected emissions. Thus, an area of high-intensity emission may be displayed as a red pixel and an area of low-intensity emission as a blue pixel. As another example, detected emissions of one wavelength at a particular sub-area of the substrate may be represented as a red pixel, and emissions of a second wavelength detected at another sub-area may be represented by an adjacent blue pixel. Many other display schemes are known. Various techniques may be applied for identifying the data representing detected emissions and separating them from background information. For example, U.S. Pat. No. 6,090,555, and U.S. patent application Ser. No. 10/197,369, titled “System, Method, and Computer Program Product for Scanned Image Alignment” filed Jul. 17, 2002, which are both hereby incorporated by reference herein in their entireties for all purposes, describe various of these techniques. In a particular implementation, scanner 150 may identify one or more labeled targets. For instance, sample of a first target may be labeled with a first dye (an example of what may more generally be referred to hereafter as an “emission label”) that fluoresces at a particular characteristic frequency, or narrow band of frequencies, in response to an excitation source of a particular frequency. A second target may be labeled with a second dye that fluoresces at a different characteristic frequency. The excitation source for the second dye may, but need not, have a different excitation frequency than the source that excites the first dye, e.g., the excitation sources could be the same, or different, lasers. The target samples may be mixed and applied to the probe arrays, and conditions may be created conducive to hybridization reactions, all in accordance with known techniques.

Probe Arrays 152: Various techniques and technologies may be used for synthesizing dense arrays of biological materials on or in a substrate or support. For example, Affymetrix® GeneChip® arrays are synthesized in accordance with techniques sometimes referred to as VLSIPS™ (Very Large Scale Immobilized Polymer Synthesis) technologies. Some aspects of VLSIPS™ and other microarray manufacturing technologies are described in U.S. Pat. Nos. 5,424,186; 5,143,854; 5,445,934; 5,744,305; 5,831,070; 5,837,832; 6,022,963; 6,083,697; 6,291,183; 6,309,831; and 6,310,189, all of which are hereby incorporated by reference in their entireties for all purposes. The probes of these arrays in some implementations consist of nucleic acids that are synthesized by methods including the steps of activating regions of a substrate and then contacting the substrate with a selected monomer solution. As used herein, nucleic acids may include any polymer or oligomer of nucleosides or nucleotides (polynucleotides or oligonucleotides) that include pyrimidine and/or purine bases, preferably cytosine, thymine, and uracil, and adenine and guanine, respectively. Nucleic acids may include any deoxyribonucleotide, ribonucleotide, and/or peptide nucleic acid component, and/or any chemical variants thereof such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. Probes of other biological materials, such as peptides or polysaccharides as non-limiting examples, may also be formed. For more details regarding possible implementations, see U.S. Pat. No. 6,156,501, which is hereby incorporated by reference herein in its entirety for all purposes.

A system and method for efficiently synthesizing probe arrays using masks is described in U.S. patent application Ser. No. 09/824,931; a system and method for a rapid and flexible microarray manufacturing and online ordering system is described in U.S. Provisional Patent Application, Serial No. 60/265,103; and systems and methods for optical photolithography without masks are described in U.S. Pat. No. 6,271,957 and in U.S. patent application Ser. No. 09/683,374, all of which are hereby incorporated by reference herein in their entireties for all purposes.

The probes of synthesized probe arrays typically are used in conjunction with biological target molecules of interest, such as cells, proteins, genes or EST's, other DNA sequences, or other biological elements. More specifically, the biological molecule of interest may be a ligand, receptor, peptide, nucleic acid (oligonucleotide or polynucleotide of RNA or DNA), or any other of the biological molecules listed in U.S. Pat. No. 5,445,934 (incorporated by reference above) at column 5, line 66 to column 7, line 51. For example, if transcripts of genes are the interest of an experiment, the target molecules would be the transcripts. Other examples include protein fragments, small molecules, etc. Target nucleic acid refers to a nucleic acid (often derived from a biological sample) of interest. Frequently, a target molecule is detected using one or more probes. As used herein, a probe is a molecule for detecting a target molecule. A probe may be any of the molecules in the same classes as the target referred to above. As non-limiting examples, a probe may refer to a nucleic acid, such as an oligonucleotide, capable of binding to a target nucleic acid of complementary sequence through one or more types of chemical bonds, usually through complementary base pairing, usually through hydrogen bond formation. As noted above, a probe may include natural (i.e. A, G, U, C, or T) or modified bases (7-deazaguanosine, inosine, etc.). In addition, the bases in probes may be joined by a linkage other than a phosphodiester bond, so long as the bond does not interfere with hybridization. Thus, probes may be peptide nucleic acids in which the constituent bases are joined by peptide bonds rather than phosphodiester linkages. Other examples of probes include antibodies used to detect peptides or other molecules, any ligands for detecting its binding partners. When referring to targets or probes as nucleic acids, it should be understood that these are illustrative embodiments that are not to limit the invention in any way.

The samples or target molecules of interest (hereafter, simply targets) are processed so that, typically, they are spatially associated with certain probes in the probe array. For example, one or more tagged targets are distributed over the probe array. In accordance with some implementations, some targets hybridize with probes and remain at the probe locations, while non-hybridized targets are washed away. These hybridized targets, with their tags or labels, are thus spatially associated with the probes. The hybridized probe and target may sometimes be referred to as a probe-target pair. Detection of these pairs can serve a variety of purposes, such as to determine whether a target nucleic acid has a nucleotide sequence identical to or different from a specific reference sequence. See, for example, U.S. Pat. No. 5,837,832, referred to and incorporated above. Other uses include gene expression monitoring and evaluation (see, e.g., U.S. Pat. Nos. 5,800,992 and 6,040,138, and International Application No. PCT/US98/15151, published as WO99/05323), genotyping (U.S. Pat. No. 5,856,092), or other detection of nucleic acids, all of which are hereby incorporated by reference herein in their entireties for all purposes.

Other techniques exist for depositing probes on a substrate or support. For example, “spotted arrays” are commercially fabricated, typically on microscope slides. These arrays consist of liquid spots containing biological material of potentially varying compositions and concentrations. For instance, a spot in the array may include a few strands of short oligonucleotides in a water solution, or it may include a high concentration of long strands of complex proteins. The Affymetrix® 417™ Arrayer and 427™ Arrayer are devices that deposit densely packed arrays of biological materials on microscope slides in accordance with these techniques. Aspects of these, and other, spot arrayers are described in U.S. Pat. Nos. 6,040,193 and 6,136,269; in U.S. patent application Ser. No. 09/683,298, in U.S. Provisional Patent Application No. 60/288,403; and in PCT Application No. PCT/US99/00730 (International Publication Number WO 99/36760), all of which are hereby incorporated by reference in their entireties for all purposes. Other techniques for generating spotted arrays also exist. For example, U.S. Pat. No. 6,040,193 to Winkler, et al. is directed to processes for dispensing drops to generate spotted arrays. The '193 patent, and U.S. Pat. No. 5,885,837 to Winkler, also describe the use of micro-channels or micro-grooves on a substrate, or on a block placed on a substrate, to synthesize arrays of biological materials. These patents further describe separating reactive regions of a substrate from each other by inert regions and spotting on the reactive regions. The '193 and '837 patents are hereby incorporated by reference in their entireties. Another technique is based on ejecting jets of biological material to form a spotted array. Other implementations of the jetting technique may use devices such as syringes or piezo electric pumps to propel the biological material. It will be understood that the foregoing are non-limiting examples of techniques for synthesizing, depositing, or positioning biological material onto or within a substrate. For example, although a planar array surface is preferred in some implementations of the foregoing, a probe array may be fabricated on a surface of virtually any shape or even a multiplicity of surfaces. Arrays may comprise probes synthesized or deposited on beads, fibers such as fiber optics, glass or any other appropriate substrate, see U.S. Pat. Nos. 6,361,947, 5,770,358, 5,789,162, 5,708,153 and 5,800,992, all of which are hereby incorporated in their entireties for all purposes. Arrays may be packaged in such a manner as to allow for diagnostics or other manipulation of in an all inclusive device, see for example, U.S. Pat. Nos. 5,856,174 and 5,922,591 incorporated in their entireties by reference for all purposes.

To ensure proper interpretation of the term “probe” as used herein, it is noted that contradictory conventions exist in the relevant literature. The word “probe” is used in some contexts to refer not to the biological material that is synthesized on a substrate or deposited on a slide, as described above, but to what has been referred to herein as the “target.” To avoid confusion, the term “probe” is used herein to refer to probes such as those synthesized according to the VLSIPS™ technology; the biological materials deposited so as to create spotted arrays; and materials synthesized, deposited, or positioned to form arrays according to other current or future technologies. Thus, microarrays formed in accordance with any of these technologies may be referred to generally and collectively hereafter for convenience as “probe arrays.” Moreover, the term “probe” is not limited to probes immobilized in array format. Rather, the functions and methods described herein may also be employed with respect to other parallel assay devices. For example, these functions and methods may be applied with respect to probe-set identifiers that identify probes immobilized on or in beads, optical fibers, or other substrates or media.

In many implementations probes are able to detect the expression of corresponding genes or EST's by detecting the presence or abundance of mRNA transcripts present in the target. This detection may, in turn, be accomplished in some implementations by detecting labeled cRNA that is derived from cDNA derived from the mRNA in the target.

Other implementations of probes may be designed to interrogate the sequence composition of DNA such as for instance, probes that interrogate single nucleotide polymorphisms (hereafter referred to as SNP's) or probes that interrogate the nucleotide composition at a specific sequence position. In some implementations, a process that is commonly referred to as polymerase chain reaction (hereafter referred to as PCR) may be used to amplify selected regions of DNA. An individual probe is capable of detecting a specific nucleic acid at a specific sequence position within a PCR product or DNA sequence. In general, a group of probes, sometimes referred to as a probe set, contains sub-sequences in unique regions of the transcripts and does not correspond to a full gene sequence.

For example, one possible embodiment of SNP probes may be present on the array so that each SNP is represented by a collection of probes. The array may comprise between 8 and 80 probes for each SNP. In one embodiment the collection comprises about 56 probes for each SNP. The probes may be present in sets of 8 probes that correspond to a perfect match or PM probe for each of two alleles, a mismatch or MM probe for each of 2 alleles, and the corresponding probes for the opposite strand. So for each allele there may be a perfect match, a perfect mismatch, an antisense match and an antisense mismatch probe. The polymorphic position may be the central position of the probe region, for instance, the probe region may be 25 nucleotides and the polymorphic allele may be in the middle with 12 nucleotides on either side. In other probe sets the polymorphic position may be offset from the center. In the present example, the polymorphic position may be from 1 to 5 bases from the central position on either the 5′ or 3′ side of the probe. The interrogation position, which may be changed in the mismatch probes, may remain at the center position. For instance, an embodiment may include 56 probes for each SNP: the 8 probes corresponding to the polymorphic position at the center or 0 position and 8 probes for the polymorphic position at each of the following positions −4, −2, −1, +1, +3 and +4 relative to the central or 0 position.

Further details regarding the design and use of probes and probe sets are provided in U.S. Pat. No. 6,188,783; in PCT Application Serial No. PCT/US 01/02316, filed Jan. 24, 2001; in U.S. patent application Ser. Nos. 09/721,042, 09/718,295, 09/745,965, and 09/764,324; and in U.S. Provisional Patent Application Serial No. 60/470,475, titled “Methods for Genotyping Polymorphisms in Humans”, filed May 14, 2003, all of which are hereby incorporated herein by reference in their entireties for all purposes.

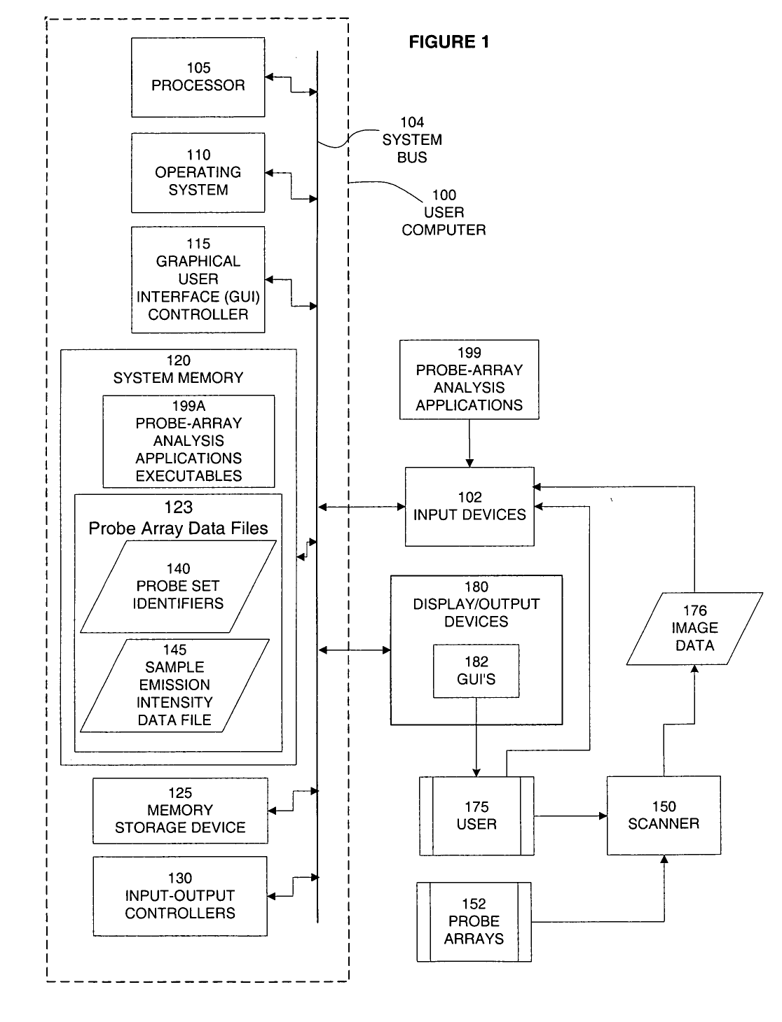

Probe Set Identifiers 140: Probe-set identifiers typically come to the attention of a user, represented by user 175 of FIG. 1, as a result of experiments conducted on probe arrays. For example, user 175 may select probe-set identifiers that identify microarray probe sets capable of enabling detection of the expression of mRNA transcripts from corresponding genes or EST's of particular interest. As is well known in the relevant art, an EST is a fragment of a gene sequence that may not be fully characterized, whereas a gene sequence generally is complete and fully characterized. The word “gene” is used generally herein to refer both to full size genes of known sequence and to computationally predicted genes. In some implementations, the specific sequences detected by the arrays that represent these genes or EST's may be referred to as, “sequence information fragments (SIF's)” and may be recorded in what may be referred to as a “SIF file.” In particular implementations, a SIF is a portion of a consensus sequence that has been deemed to best represent the mRNA transcript from a given gene or EST. The consensus sequence may have been derived by comparing and clustering EST's, and possibly also by comparing the EST's to genomic sequence information. A SIF is a portion of the consensus sequence for which probes on the array are specifically designed. With respect to the operations of sequence data manager 323 of the particular implementation described herein, it is assumed with respect to some aspects that some microarray probe sets may be designed to detect the sequence composition of DNA from PCR amplified fragments.

As was described above, the term “probe set” refers in some implementations to one or more probes from an array of probes on a microarray. For example, in an Affymetrix® GeneChip® probe array, in which probes are synthesized on a substrate, a probe set may consist of 30 or 40 probes, half of which typically are controls. These probes collectively, or in various combinations of some or all of them, are deemed to be indicative of the expression of a gene or EST. In a spotted probe array, one or more spots may similarly constitute a “probe set.”

The term “probe-set identifiers” is used broadly herein in that a number of types of such identifiers are possible and may be included within the meaning of this term in various implementations. One type of probe-set identifier is a name, number, or other symbol that is assigned for the purpose of identifying a probe set. This name, number, or symbol may be arbitrarily assigned to the probe set by, for example, the manufacturer of the probe array. A user may select this type of probe-set identifier by, for example, highlighting or typing the name. Another type of probe-set identifier as intended herein is a graphical representation of a probe set. For example, dots may be displayed on a scatter plot or other diagram wherein each dot represents a probe set, as described for example in U.S. Pat. No. 6,420,108, which is hereby incorporated herein in its entirety for all purposes. Typically, the dot's placement on the plot represents the intensity of the signal from hybridized, tagged, targets (as described in greater detail below) in one or more experiments. In these cases, a user may select a probe-set identifier by clicking on, drawing a loop around, or otherwise selecting one or more of the dots. In another example, user 175 may select a probe-set identifier by selecting a row or column in a table or spreadsheet that correlates probe sets with accession numbers and other genomic information.

Yet another type of probe-set identifier, as that term is used herein, includes a nucleotide or amino acid sequence. For example, it is illustratively assumed that a particular SIF is a unique sequence of 500 bases that is a portion of a consensus sequence or exemplar sequence gleaned from EST and/or genomic sequence information. It further is assumed that one or more probe sets are designed to represent the SIF. A user who specifies all or part of the 500-base sequence thus may be considered to have specified all or some of the corresponding probe sets.

As a further example with respect to a particular implementation, a user may specify a portion of the 500-base sequence noted above, which may be unique to that SIF, or, alternatively, may also identify another SIF, EST, cluster of EST's, consensus sequence, and/or gene or protein. The user thus specifies a probe-set identifier for one or more genes or EST's. In another variation, it is illustratively assumed that a particular SIF is a portion of a particular consensus sequence. It is further assumed that a user specifies a portion of the consensus sequence that is not included in the SIF but that is unique to the consensus sequence or the gene or EST's the consensus sequence is intended to represent. In that case, the sequence specified by the user is a probe-set identifier that identifies the probe set corresponding to the SIF, even though the user-specified sequence is not included in the SIF. Parallel cases are possible with respect to user specifications of partial sequences of EST's and genes or EST's, as those skilled in the relevant art will now appreciate.

A further example of a probe-set identifier is an accession number of a gene or EST. Gene and EST accession numbers are publicly available. A probe set may therefore be identified by the accession number or numbers of one or more EST's and/or genes corresponding to the probe set. The correspondence between a probe set and EST's or genes may be maintained in a suitable database from which the correspondence may be provided to the user. Similarly, gene fragments or sequences other than EST's may be mapped (e.g., by reference to a suitable database) to corresponding genes or EST's for the purpose of using their publicly available accession numbers as probe-set identifiers. For example, a user may be interested in product or genomic information related to a particular SIF that is derived from EST-1 and EST-2. The user may be provided with the correspondence between that SIF (or part or all of the sequence of the SIF) and EST-1 or EST-2, or both. To obtain product or genomic data related to the SIF, or a partial sequence of it, the user may select the accession numbers of EST-1, EST-2, or both.

In some embodiments, probe set identifiers may also include those associated with genotyping applications. Such genotyping applications may for example, include the identification of single nucleotide polymorphisms or regions of genomic sequence that may, for instance, include a chromosome, whole genome, or other type of genomic sequence known to those of ordinary skill in the related art. For example, a probe array may interrogate a plurality of SNP's where each SNP may be used as a probe set identifier for one or more probe sets. Alternatively, a region of genomic sequence may also identify one or more probe sets. Also, in the present example SNP identifiers such as, for instance those used by dbSNP, or identifiers associated with genomic sequence may also be used as probe set identifiers.

Additional examples of probe-set identifiers include one or more terms that may be associated with the annotation of one or more gene or EST sequences, where the gene or EST sequences may be associated with one or more probe sets. For convenience, such terms may hereafter be referred to as “annotation terms” and will be understood to potentially include, in various implementations, one or more words, graphical elements, characters, or other representational forms that provide information that typically is biologically relevant to or related to the gene or EST sequence. Associations between the probe-set identifier terms and gene or EST sequences may be stored in a database such as a local genomic database, or they may be transferred from one or more remote databases. Examples of such terms associated with annotations include those of molecular function (e.g. transcription initiation), cellular location (e.g. nuclear membrane), biological process (e.g. immune response), tissue type (e.g. kidney), or other annotation terms known to those in the relevant art.

Probe-Array Analysis Applications 199: Generally, a human being may inspect a printed or displayed image constructed from the data in an image file and may identify those cells that are bright or dim, or are otherwise identified by a pixel characteristic (such as color). However, it frequently is desirable to provide this information in an automated, quantifiable, and repeatable way that is compatible with various image processing and/or analysis techniques. For example, the information may be provided for processing by a computer application that associates the locations where hybridized targets were detected with known locations where probes of known identities were synthesized or deposited. Other methods include tagging individual synthesis or support substrates (such as beads) using chemical, biological, electromagnetic transducers or transmitters, and other identifiers. Information such as the nucleotide or monomer sequence of target DNA or RNA may then be deduced. Techniques for making these deductions are described, for example, in U.S. Pat. No. 5,733,729, which hereby is incorporated by reference in its entirety for all purposes, and in U.S. Pat. No. 5,837,832, noted and incorporated above.

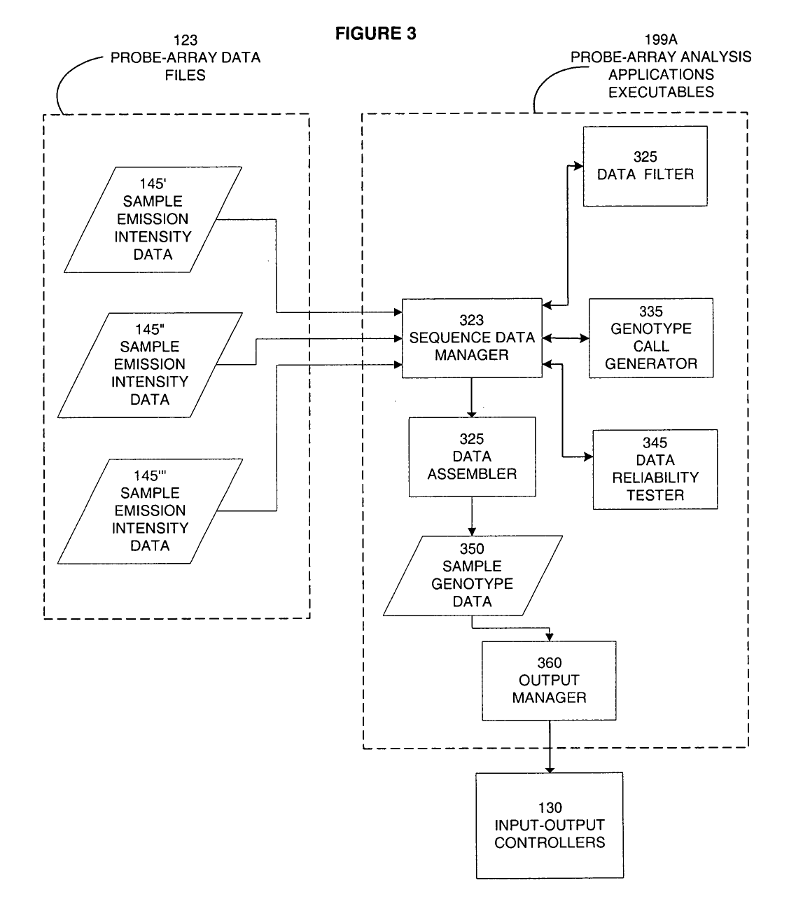

A variety of computer software applications are commercially available for controlling scanners (and other instruments related to the hybridization process, such as hybridization chambers), and for acquiring and processing the image files provided by the scanners. Examples are the Jaguar™ application from Affymetrix, Inc., aspects of which are described in PCT Application PCT/US 01/26390 and in U.S. patent application Ser. Nos. 09/681,819, 09/682,071, 09/682,074, 09/682,076, and 10/197,369, and the Microarray Suite application from Affymetrix, aspects of which are described in U.S. Provisional Patent Applications, Serial Nos. 60/220,587, 60/220,645 and 60/312,906, and in U.S. patent application Ser. No. 10/219,882, all of which are hereby incorporated herein by reference in their entireties for all purposes. For example, image data in image data file 176 may be operated upon to generate intermediate results such as so-called cell intensity files (*.cel) and chip files (*.chp), generated by Microarray Suite or spot files (*.spt) generated by Jaguar™ software. For convenience, the terms “file” or “data structure” may be used herein to refer to the organization of data, or the data itself generated or used by executables 199A and executable counterparts of other applications. However, it will be understood that any of a variety of alternative techniques known in the relevant art for storing, conveying, and/or manipulating data may be employed, and that the terms “file” and “data structure” therefore are to be interpreted broadly. In the illustrative case in which image data file 176 is derived from a GeneChip® probe array, and in which Microarray Suite may generate one or more sets of data or data files contained in probe array data files 123. FIG. 3 further illustrates an example of data files 123 that may include sample emission intensity data 145′, 145″, and 145′″. Each of data 145 may contain emission intensity data for each probe feature disposed upon a probe array. In the present example data 145′ may correspond to a particular probe array type where an experimental sample has been tested. Additionally, data 145″ and 145′″ may correspond to the same probe array type where different experimental samples have been used that may allow for the comparison between experimental samples. Those of ordinary skill in the related art will appreciate that each of files 145 may include one or more sets of data or data files that may correspond to one or more experimental samples.

Files 145 may contain, for each probe feature scanned by scanner 150, a single value representative of the intensities of pixels measured by scanner 150 for that probe feature. Thus, this value is a measure of the abundance of tagged cRNA's present in the target that hybridized to the corresponding probe feature. Many such cRNA's may be present in each probe feature, as a probe feature on a GeneChip® probe array may include, for example, millions of oligonucleotides designed to detect the cRNA's. The resulting data stored in the chip file may include degrees of hybridization, absolute and/or differential (over two or more experiments) expression, genotype comparisons, detection of polymorphisms and mutations, and other analytical results. In another example, in which executables 199A includes image data from a spotted probe array, the resulting spot file includes the intensities of labeled targets that hybridized to probes in the array. Further details regarding cell files, chip files, and spot files are provided in U.S. Provisional Patent Application Nos. 60/220,645, 60/220,587, and 60/226,999, incorporated by reference above.

In the present example, in which executables 199A include Affymetrix® Microarray Suite, the chip file is derived from analysis of the cell file combined in some cases with information derived from library files. Laboratory or experimental data may also be provided to the software for inclusion in the chip file. For example, an experimenter and/or automated data input devices or programs may provide data related to the design or conduct of experiments. As a non-limiting example, the experimenter may specify an Affymetrix catalogue or custom chip type (e.g., Human Genome U95Av2 chip) either by selecting from a predetermined list presented by Microarray Suite or by scanning a bar code related to a chip to read its type. Also, this information may be automatically read. For example, a bar code (or other machine-readable information such as may be stored on a magnetic strip, in memory devices of a radio transmitting module, or stored and read in accordance with any of a variety of other known techniques) may be affixed to the probe array, a cartridge, or other housing or substrate coupled to or otherwise associated with the array. The machine-readable information may automatically be read by a device (e.g., a 1-D or 2-D bar code reader) incorporated within the scanner, an autoloader associated with the scanner, an autoloader movable between the scanner and other instruments, and so on. In any of these cases, Microarray Suite may associate the chip type, or other identifier, with various scanning parameters stored in data tables. The scanning parameters may include, for example, the area of the chip that is to be scanned, the starting place for a scan, the location of chrome borders on the chip used for auto-focusing, the speed of the scan, a number of scan repetitions, the wavelength or intensity of laser light to be used in reading the chip, and so on. Rather than storing this data in data tables, some or all of it may be included in the machine-readable information coupled or associated with the probe arrays. Other experimental or laboratory data may include, for example, the name of the experimenter, the dates on which various experiments were conducted, the equipment used, the types of fluorescent dyes used as labels, protocols followed, and numerous other attributes of experiments.

As noted, executables 199A may apply some of this data in the generation of intermediate results. For example, information about the dyes may be incorporated into determinations of relative expression. Other data, such as the name of the experimenter, may be processed by executables 199A or may simply be preserved and stored in files or other data structures. Any of these data may be provided, for example over a network, to a laboratory information management server computer, configured to manage information from large numbers of experiments. A data analysis program may also generate various types of plots, graphs, tables, and other tabular and/or graphical representations of analytical data. As will be appreciated by those skilled in the relevant art, the preceding and following descriptions of files generated by executables 199A are exemplary only, and the data described, and other data, may be processed, combined, arranged, and/or presented in many other ways.

The processed image files produced by these applications often are further processed to extract additional data. In particular, data-mining software applications often are used for supplemental identification and analysis of biologically interesting patterns or degrees of hybridization of probe sets. An example of a software application of this type is the Affymetrix® Data Mining Tool, described in U.S. patent application Ser. No. 09/683,980, which is hereby incorporated herein by reference in its entireties for all purposes. Software applications also are available for storing and managing the enormous amounts of data that often are generated by probe-array experiments and by the image-processing and data-mining software noted above. An example of these data-management software applications is the Affymetrix® Laboratory Information Management System (LIMS). In addition, various proprietary databases accessed by database management software, such as the Affymetrix® EASI (Expression Analysis Sequence Information) database and database software, provide researchers with associations between probe sets and gene or EST identifiers.

For convenience of reference, these types of computer software applications (i.e., for acquiring and processing image files, data mining, data management, and various database and other applications related to probe-array analysis) are generally and collectively represented in FIG. 1 as probe-array analysis applications 199. FIG. 1 illustratively shows applications 199 stored for execution (as executable code 199A corresponding to applications 199) in system memory 120 of user computer 100.

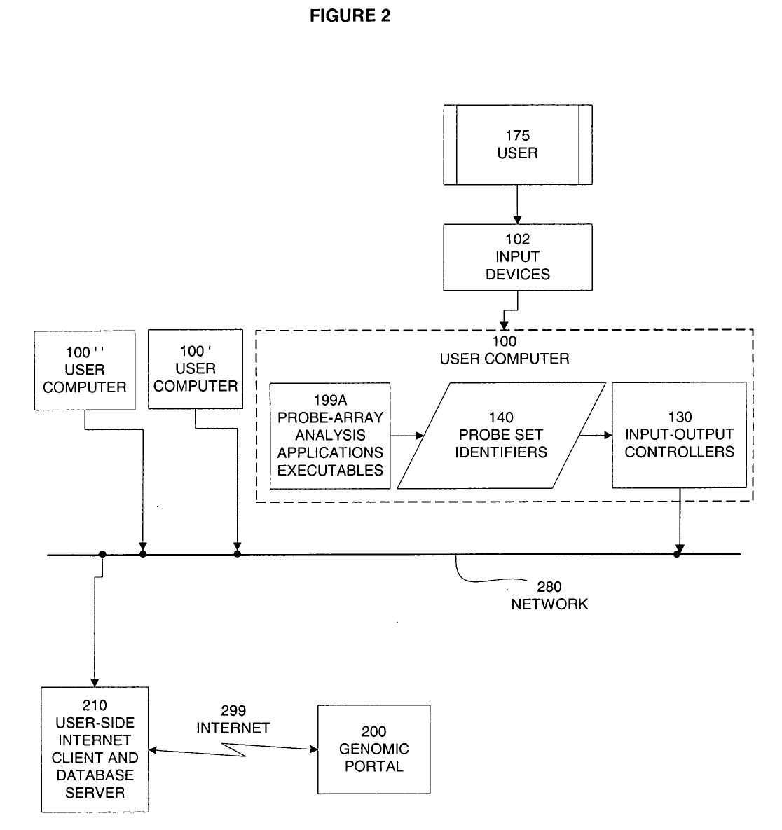

As will be appreciated by those skilled in the relevant art, it is not necessary that applications 199 be stored on and/or executed from computer 100; rather, some or all of applications 199 may be stored on and/or executed from an applications server or other computer platform to which computer 100 is connected in a network. For example, it may be particularly advantageous for applications involving the manipulation of large databases to be executed from a database server such as user-side internet client and database server 210 of FIG. 2. Alternatively, LIMS, DMT, and/or other applications may be executed from computer 100. But some or all of the databases upon which those applications operate may be stored for common access on server 210 (perhaps together with a database management program, such as the Oracle® 8.0.5 database management system from Oracle Corporation). Such networked arrangements may be implemented in accordance with known techniques using commercially available hardware and software, such as those available for implementing a local-area network or wide-area network. A local network is represented as network 280 by the connection of user computer 100 to database server 210 (and to a user-side Internet client, which is illustrated in FIG. 2 as the same computer but need not be). The connections of network 280 could include a network cable, wireless network, or other means of networking known to those in the related art. Similarly, scanner 150 (or multiple scanners) may be made available to a network of users over a network cable both for purposes of controlling scanner 150 and for receiving data input from it.

In some implementations, it may be convenient for user 175 to group probe-set identifiers for batch transfer of information or to otherwise analyze or process groups of probe sets together. For example, as described below, user 175 may wish to obtain annotation information related to one or more probe sets identified by their respective probe set identifiers 140. Rather than obtaining this information serially, user 175 may group probe sets together for batch processing. Various known techniques may be employed for associating probe set identifiers 140, or data related to those identifiers, together. For instance, user 175 may generate a tab delimited *.txt file including a list of probe set identifiers 140 for batch processing. This file or another file or data structure for providing a batch of data (hereafter referred to for convenience simply as a “batch file”), may be any kind of list, text, data structure, or other collection of data in any format. The batch file may also specify what kind of information user 175 wishes to obtain with respect to all, or any combination of, the identified probe sets. In some implementations, user 175 may specify a name or other user-specified identifier to represent the group of probe-set identifiers specified in the text file or otherwise specified by user 175. This user-specified identifier may be stored by one of executables 199A, so that user 175 may employ it in future operations rather than providing the associated probe-set identifiers in a text file or other format. Thus, for example, user 175 may formulate one or more queries associated with a particular user-specified identifier, resulting in a batch transfer of information from portal 200 to user 175 related to the probe-set identifiers that user 175 has associated with the user-specified identifier. Alternatively, user 175 may initiate a batch transfer by providing the text file of probe-set identifiers. In any of these cases, user 175 may provide information, such as laboratory or experimental information, related to a number of probe sets by a batch operation rather than serial ones. The probe sets may be grouped by experiments, by similarity of probe sets (e.g., probe sets representing genes having similar annotations, such as related to transcription regulation), or any other type of grouping. For example, user 175 may assign a user-specified identifier (e.g., “experiments of January 1”) to a series of experiments and submit probe-set identifiers in user-selected categories (e.g., identifying probe sets that were up-regulated by a specified amount).

Similarly, user 175 may use probe set identifiers 140 for the design of custom probe arrays. User 175 may want to use probe arrays with a particular combination of probe sets disposed upon them that may not be available as a commercial product. Additionally, a user may wish to use probe sets that are not available. In both cases the user may submit a plurality of probe set identifiers and other selected specifications for the custom production of probe sets, and/or probe arrays. User 175 may electronically submit probe set identifiers individually or by batch transfer as previously described. The methods electronic submission could include submission by e-mail, or other methods of electronic submission known to those of ordinary skill in the related art. One such example is illustrated in FIG. 2 where the user may submit the probe set identifiers via Internet 299 to genomic portal 200. Portal 200 may interactively provide the user with information that could include a confirmation that the plurality of probe set identifiers had been received, expected shipping dates, price quotes, or other information that might be of interest to the user. In the present example, portal 200 is specifically enabled to receive a plurality of probe set identifiers for probe array design. Portal 200 could for instance be a web portal provided by Affymetrix®, Inc.

Further details regarding the submission of probe set identifiers for custom array design are described in U.S. Provisional Patent Application 60/310,298, and U.S. patent application Ser. No. 10/036,559, each of which is hereby incorporated by reference herein in their entireties for all purposes.

Sequence Data Manager 323: Another element of the illustrated implementation of probe array analysis executables 199A may include sequence data manager 323. In one embodiment sequence data manager 323 may manage the functions of analyzing the emission intensity values contained within probe array data files 123, illustrated in FIG. 3 as data 145′, data 145″, and data 145′″. In the illustrated implementation, each of data 145 may represent emission intensity data from a probe array experiment conducted on an individual sample. Data manager 323 may concurrently analyze a plurality of samples that could, for instance, include 200 or more samples.

In one embodiment manager 323 may implement what are referred to as genotyping algorithms for the analysis of emission intensity data that, for example, may be derived from probe arrays designed to interrogate a plurality of selected DNA sequences. The probe arrays may in some implementations require many copies of a selected DNA sequence in order to obtain reliable data. Many copies of a DNA sequence may be produced by a process that is commonly referred to by those of ordinary skill in the related art as Polymerase Chain Reaction (hereafter referred to as PCR). The term “PCR” as described herein generally refers to methods that “amplify” (i.e. make many copies of), a particular DNA sequence or other selected sequence of interest.

In some implementations data manager 323 may employ one or more genotyping algorithms that may be enabled to identify the composition of nucleic acid bases of a selected DNA sequence from scanned probe array data, and may sometimes be referred to as sequencing or resequencing. Additionally, manager 323 may employ one or more of the algorithms to identify specific variations within a specified sequence such as, for instance, what are referred to as single nucleotide polymorphisms (hereafter referred to as SNP's). For example, one type of algorithm could include the CustomSeq™ algorithm from Affymetrix, Inc. The CustomSeq™ algorithm may be used to determine the nucleic acid composition for each sequence position of a selected DNA sequence. In the present example, the algorithm may use the emission intensity data values from probe sets disposed on probe arrays designed to interrogate specific regions genomic DNA or other type of sequences. The regions of genomic DNA may include sequences measured in bases, kilobases, megabases, centimorgans, chromosomes, or genomes. The emission intensity data values may be contained within one or more data files that could for instance include *.cel file.

In one possible implementation, manager 323 may implement the algorithm in a number of steps as illustrated in FIG. 7. As illustrated in step 710, manager 323 may employ data filters 325 to identify unreliable data or adjust what is referred to as the variance of the emission intensities that may approach the limits of detection. The term “variance” as used herein generally refers to a value that includes a measure of the dispersion of data. For example, those of ordinary skill in the related art will appreciate that variance may be defined by the following equation:

$${σ}^{2} = \frac{\sum {( X - \overline{X} )}^{2}}{n - 1}$$

In the present example, X is equal to a particular value that could for instance be an emission intensity value for a probe feature. Similarly, \overline{X} is equal to the mean of all X values and n is equal to the total number of values.

In the some implementations data filters 325 may use the emission intensity values of one or more probe sets associated with an experimental sample to determine whether to call a sequence position as a no call (n) or to make an adjustment to the variance value corresponding to the experimental probe array. For example, data filters 325 may take into account emission intensity values associated with two probe sets that represent the same position in the genomic sequence and sometimes referred to as RAS1 and RAS2. For instance, one probe set may be designed to interrogate a sequence position on the coding or forward strand, and another probe set may be designed to interrogate the corresponding sequence position on the non-coding or reverse strand.

As illustrated in step 710, data filters 325 may filter emission intensity data associated with each of data 145 for certain categories of characteristics that could include no signal, weak signal, saturated signal, or high signal to noise ratio. In some instances data filers 325 may rule a sequence position as a no call (n) if the emission intensity data does not meet one or more criteria associated with each of the categories, or filters 325 may adjust one or more variance values based, at least in part, upon measured intensity values that approach the limits of the detector. For example, each sequence position associated with a sample that is ruled as a no call (n) may be recorded in sample genotype call data 350.

The no signal category could include criteria such as a threshold value for what may be referred to as the mean intensity value. Each probe feature of a probe set may have a unique mean intensity value, and may be defined as the mean value of the emission intensity values for all pixels within the probe feature. The threshold value could include a pre-defined value that may be a value that within two standard deviations of zero. Alternatively the threshold value could be a value that the user selects. The term “standard deviation” as used herein generally refers to a value that is the square root of the variance. In the present implementation, the standard deviation value may be derived from emission intensity data from each of the probe features of the one or more probe sets for a sequence position from one or more samples. Alternatively the standard deviation value may be derived from a subset of one or more probe features such as for instance, the base composition of a feature (i.e. A, C, G, or T), from a probe set for a particular strand (i.e. coding or non-coding strand), or from all probe sets of the probe array. If, for example, the mean intensity value for any probe feature of a probe set is below the threshold value then the call assigned to the corresponding sequence position will be no call (n). Otherwise the criteria have been satisfied for the category and a call may not be assigned by filters 325.

The weak signal category could include criteria such as a threshold value for what may be referred to as the highest mean intensity value. The highest mean intensity value may be defined as the mean intensity value for a probe feature that is higher than all other mean intensity values of probe features in a probe set. The threshold value could include a pre-defined value such as, for instance, a value equal to a 20 fold decrease from the average highest mean intensities for all probe sets from the same strand (i.e. coding or non-coding strands). Alternatively, the threshold value may include a value that is selected by the user. If, for example, the highest mean intensity value for a probe set is below the threshold value then the call assigned to the corresponding sequence position will be no call (n). Otherwise the criteria have been satisfied for the category and a call would not be assigned by filters 325.

The saturation category could include criteria such as a threshold value that a plurality of probe features of a probe set may need to fail in order for a no call (n) assignment to be made. The threshold value may include a pre-defined value such as, for instance, a value that is two standard deviations below 43,000. In some implementations, the 43,000 value may be associated with the maximum emission intensity value that is at the limit of detection for a scanning system. Those of ordinary skill in the related art will appreciate that other values may be used that are representative of the detection limit of each specific system. As in the previous categories the user may also select the threshold value. Additionally, the standard deviation value may be the same as that used for the no signal category, or alternatively may be different being derived from another set of emission intensity values. Other criteria for the category may also include a maximum number of probe features that do not satisfy the threshold value criteria in order to assign a no call (n) to the sequence position. For example, a sequence position may correspond to a chromosome that may be in what is referred to as a haploid state (i.e. generally a haploid state refers to the presence of a single chromosome, and a diploid state refers to a pair of similar chromosomes). If two or more probe features of the probe set have mean intensity values greater than the threshold value (i.e. 43000) then the sequence position is assigned as a no call (n). Also in the present example, if the sequence position corresponds to a diploid state, then three or more features must be higher than the threshold value for a no call (n) assignment to be made by filters 325.

The signal to noise ratio category could include criteria such as a threshold value for what is referred to as the signal to noise ratio. The term “signal to noise ratio” as used herein generally refers to the ratio of emission intensity values from the signal generated from hybridized probes to the emission intensity values from what is referred to as noise. Noise could include the fluorescent emissions generated from residual unbound sample, the non-specific binding of sample to probe features, or other processes that may generate fluorescent emissions that do not include the specific binding of sample to probe features. The threshold may include a pre-defined value such as, for instance 20, or a user selectable value. In some implementations, if the signal to noise ratio exceeds the threshold value, filters 325 may adjust one or more parameters such as, for instance variance, so that the signal to noise ration is equal to the threshold value. For example, if the signal to noise ratio for all probe sets of a given sample is greater than 20, then the variance for all probe sets of the sample may be set at so that the signal to noise ratio is equal to 20. In an alternative example, the signal to noise ratio within a probe set, or the one or more probe sets that correspond to a sequence position may be greater than the threshold value. In such an example the variance that corresponds to the one or more probe sets may be set so that the signal to noise ratio of the one or more probe sets is equal to the threshold value.

Sequence data manager 323 then forwards the filtered emission intensity data to genotype call generator 335 to perform the next steps illustrated as step 720. The processes performed by genotype call generator 335 may be based, at least in part, upon models developed to specify the presence or absence of specific nucleic acids in each sequence position of a selected DNA sequence based, at least in part, upon detected emission intensity values for associated probe sets. In some embodiments, two different sets of models may be applied to the data based upon different assumptions. The assumptions may be based upon what may be referred to as an even background or uneven background that will be explained in more detail below.

In one embodiment, genotype call generator 335 calculates the likelihood that a particular nucleic acid fits a certain model at each sequence position. The likelihood may be determined for both the coding and non-coding strands independently, and a final likelihood for a model may then be determined by multiplying the likelihood values for the coding and non-coding strands. An equation for the log (base e) likelihood may be given by:

$$ln ( L ) = - \frac{1}{2} \sum {N}_{x} [ ln ( {\hat{σ}}_{x}^{2} ) + ( {V}_{x} + {M}_{x}^{2} - 2 {\hat{μ}}_{x} {M}_{x} + {\hat{μ}}_{x}^{2} ) / {\hat{σ}}_{x}^{2} + ln ( 2 π ) ]$$

In the illustrated equation Nₓ is the number of pixels observed in feature x, Vₓ is the observed variance for feature x, and Mₓ is the observed mean for feature x. Also μₓ is the estimated mean for feature x for the model in question and similarly σₓ² is the estimated variance for feature x. Feature x may represent a A, C, G, or T nucleotide, and the method is performed for each feature disposed upon the probe array.

For each model what are referred to as quality scores are calculated based, at least in part, upon the likelihood values. Quality scores may be calculated for each strand as well as an overall quality score. For example, the quality scores are calculated using the likelihood values of the coding strand, non-coding strand, and the overall likelihood value individually.

The quality score may be calculated by a variety of methods that could include an equation such as:

Qₛ(x)=log(Lₛ(x))−log(Lₛ(max_other))

Where Lₛ is equal to the likelihood value for the particular strand or overall value, x refers to the feature (i.e. A, C, G, or T), and max_other refers to the maximum likelihood value for a feature that is not the same as the L(x) value. For example, Q_{c}(A) may represent the quality score from the coding strand for feature A. The quality score may represent the difference between the log likelihood value of model A and the best fitting model on the same strand (i.e. coding) excluding the value for the A feature (i.e. the next highest value if the A value is the highest). If, in the present example, Q_{c}(A) is positive, then the A model may be the best fitting model on the coding strand.

In some embodiments, the models may include a no call model, homozygote models and heterozygote models. The no call model may assume that all of the probe sets have identical means and variances to the probe sets on the same strand (i.e. coding or non-coding strands), but that the means and variances of the probe sets may differ between strands. On the basis of the assumptions of the no call model the following equations for the estimated mean and variance for each strand may be:

$${\hat{μ}}_{s} ( b ) = \frac{{N}_{s} ( A ) {M}_{s} ( A ) + {N}_{s} ( C ) {M}_{s} ( C ) + {N}_{s} ( G ) {M}_{s} ( G ) + {N}_{s} ( T ) {M}_{s} ( T )}{{N}_{s} ( A ) + {N}_{s} ( C ) + {N}_{s} ( G ) + {N}_{s} ( T )}$$

$${\hat{σ}}_{s}^{2} ( b ) = \frac{\begin{matrix}{N}_{s} ( A ) ( {V}_{s} ( A ) + {M}_{s}^{2} ( A ) ) + {N}_{s} ( C ) ( {V}_{s} ( C ) + {M}_{s}^{2} ( C ) ) + \\ {N}_{s} ( G ) ( {V}_{s} ( G ) + {M}_{s}^{2} ( G ) ) + {N}_{s} ( T ) ( {V}_{s} ( T ) + {M}_{s}^{2} ( T ) )\end{matrix}}{{N}_{s} ( A ) + {N}_{s} ( C ) + {N}_{s} ( G ) + {N}_{s} ( T )} - {μ}_{s}^{2} ( b )$$

μₛ(b) and σₛ(b), in the illustrated example, represent the estimated mean and variance background intensities respectively for a particular strand that could be either the coding or non-coding strands.

The overall likelihood of the no call model may be represented as:

L(0)=L_{c}(0)Lₙ(0)

Where L_{c}(0) is the no call likelihood for the coding forward strand and Lₙ(0) is the no call likelihood for the non-coding reverse strand.

The homozygote and heterozygote models may be based similar to the no call models, but with slightly different assumptions. For example, a sample may be an A homozygote at a particular position. Thus C, G, and T on the coding forward strand are assumed to be background features and independent and identically distributed have the same mean and variance. The models for the C, G, and T bases could be represented as:

$${\hat{μ}}_{c} ( b ) = \frac{{N}_{c} ( C ) {M}_{c} ( C ) + {N}_{c} ( G ) {M}_{c} ( G ) + {N}_{c} ( T ) {M}_{c} ( T )}{{N}_{c} ( C ) + {N}_{c} ( G ) + {N}_{c} ( T )}$$

$${\hat{σ}}_{c}^{2} ( b ) = \frac{{N}_{c} ( C ) {ω}_{c} ( C ) + {N}_{c} ( G ) {ω}_{c} ( G ) + {N}_{c} ( T ) {ω}_{c} ( T )}{{N}_{c} ( C ) + {N}_{c} ( G ) + {N}_{c} ( T )}$$

Where ω_{c} for feature x may be defined as:

ω_{c}(x)=V(x)+M_{c}²(x)−2M(x)\hat{μ}_{c}(b)+\hat{μ}_{c}(b)+\hat{μ}_{c}²(b)

In the present example, feature A is assumed to have a different mean and variance. The mean and variance are statistically estimated, by a parameter estimation method known to those of ordinary skill in the related art as maximum likelihood, to be the same as the observed values.

\hat{μ}_{c}(A)=M_{c}(A)

σ_{c}²(A)=V_{c}(A)

If, in the illustrated example, \hat{μ}_{c}(A)<\hat{μ}_{c}(b) (i.e. the estimated mean for model A is less than the estimated mean of the background) then the likelihood is set to the no call model (L_{c}(A)=L_{c}(0)). Similarly, if \hat{μ}ₙ(A)<\hat{μ}ₙ(b) then Lₙ(A)=Lₙ(0).

L(A) is the overall likelihood of the A homozygote model.

L(A)=L_{c}(A)Lₙ(A)

All other homozygote models, i.e. the models for C, G, and T, are treated similarly to the above example.

The heterozygote models in the presently described implementation may only apply to diploid data for reasons that will be appreciated by those of ordinary skill in the relevant art. The heterozygote models may include A-C, A-G, A-T, C-G, C-T, and G-T. The models are again similar to the no call models, but with a different set of assumptions. For example, for an A-C heterozygote the background features on the coding forward strand for G and T are assumed to be independent and identically distributed have the same mean and intensity. Similarly features A and C on the coding reverse strand are also assumed to be independent and identically distributed. The models then reflect these assumptions.

As previously illustrated, genotype call generator 335 calculates the likelihood values and quality scores for all of the even background models. The number of models could vary depending on whether the sample in question is haploid or diploid. The terms “haploid” and “diploid” as used herein refer to the number of chromosomes that are present in a sample. Haploid generally refers to a single copy of each chromosome whereas diploid refers to the presence of two copies of each chromosome. For haploid data, the likelihood values and quality scores for a total of five models may be calculated, i.e. the no call, A, C, G, and T models. For diploid data an additional six models may be added that could include AC, AG, AT, CG, CT, and GT.

A genotype call for the sequence position may be made if one even background model fits nearly perfectly and all of the other even background models fit relatively poorly. In one possible implementation, a genotype call for a particular model may be made if the quality scores for both strands are positive (i.e. Q_{c}(x)>0 and Qₙ(x)>0), and the overall quality score is greater than a total quality threshold value (Q(x)>T_{Total}). T_{Total} could be a pre-defined value that, for example, could include a value of 5.2. T_{Total} could also be some user definable value that could for example affect the sensitivity or stringency of the genotype call.

If no even background model fits nearly perfectly, genotype call generator 335 may make a genotype call based an imperfect fit. In some implementations, there may be two quality score thresholds, T_{Total} and T_{Strand}. Both thresholds may have pre-defined values or be user definable, where the predefined threshold values may have been experimentally determined. T_{Total} may be the same value for the imperfect fit as was used for the nearly perfect fit, or alternatively may be a different value. For example, the predefined threshold values may have been experimentally determined specifically for the imperfect fit call. In the present example T_{Total} may have a predefined value of 30 and T_{Strand} could have a predefined value of −2.

Genotype call generator 335 next applies the emission intensity data from diploid samples to another set of models that may be based on a different set of assumptions. These models may be referred to as uneven background models where it may be assumed that the means and variances may not be identical uniform for all of the probe sets on a strand. For example, situations that could give rise to different means and variances could include what is referred to as cross hybridization, or unevenness of the background features. In the example of cross hybridization, a prediction may be made that assumes that all samples should exhibit the same ratio of unevenness in both means and variances across samples.

In one implementation the uneven background models could include those that account for constant ratios of unevenness between samples. Values that represent the constant ratios for the means and variances may be obtained by averaging the means and variance values at each sequence position with the same genotype call over all the samples. It will be appreciated by those of ordinary skill in the related art that the genotype calls may not be initially known for a number of sequence positions. In a some implementations, an iterative method may be used that changes the constant values as genotype calls change. The iterative method may continue until the genotype calls converge, or alternatively may proceed through a set number of iterations that could be predefined or selected by the user.

In one implementation the genotype calls for the uneven background models may be made for a nearly perfect fit and imperfect fit following the same criteria as for the even background models. Also in the presently described implementation, a genotype call may be “guessed” for a sequence position if a model fits both the coding and non-coding strand better than any other model, but does not meet the threshold requirements for an imperfect fit call. For example, a guess may be made if all the quality scores for a given model are greater than zero and the model fits better than any other model.

In the cases of both the even and uneven background models, if a model cannot be called or guessed for a given sequence position, then that position may be classified as a no call (n).

Sequence data manager 323 may then forward the genotype call results to data reliability tester 345 in order to test the reliability of the genotype calls, illustrated as step 730. In some implementations, the genotype call data must satisfy a number of criteria in order to be considered reliable. The criteria may include but are not limited to the following descriptions.

For each sequence position, at least 50% of the surrounding sequence positions must have a genotype call (i.e. A, C, G, or T) or be ruled as a no call (n). The number of surrounding sites could again be predefined or a user selected value. For example, the number of surrounding sites to be considered could have been selected by a user to be 20 that may mean that ten sites on each side of the sequence position are considered. In the present example, if there are more than 10 no calls (n) in the 20 surrounding sites, then the sequence position in question is ruled as a no call (n).

For a sequence position, if greater than 50% of the genotype calls for the same sequence position across all samples are ruled as a no call (n), then the sequence position is ruled as a no call (n). For example, each of sample emission intensity data 145′, 145″, and 145′″ may include emission intensity data for the same sequence position where each of the sets of data or data files may be associated with a particular sample. In the present example, if the genotype call for that sequence position is a no call (n) for both data 145′ and 145″, then data reliability tester 345 will assign the same sequence position as a no call (n) for data 145′″.

If two SNP's are identified within 5 sequence positions of each other, they are termed SNP doublets. For example, one SNP may be termed SNP1, and the other may be termed SNP2. Also for each SNP there may be a genotype call that is more common, and thus may be termed as the wild type call while the less common call may be termed the mutant call. Those of ordinary skill in the related art will appreciate that the previous examples are for the purposes of illustration only and should not be limiting in any way.

The rules for the determination of SNP doublets may include the following examples. If a sample is mutant for SNP1 and wild type for SNP2, and another sample is wild type at SNP one and mutant for SNP2. Then both mutant SNP calls are determined to be reliable. If a sample is mutant at SNP1 and wild type at SNP2, and all other samples that are mutant at SNP2 or have a no call (n) at SNP1. Then the SNP2 call is determined to be unreliable and all samples may be called as a no call (n) at the SNP2 sequence position. If mutants at SNP1 always occur in samples that are also mutant or no call (n) at SNP2 or vice versa. Then the SNP with the smaller number of no calls (n) is considered as reliable and the other SNP position is called as no call (n) for all samples.

Some embodiments of sequence data manager 323 may also be able to identify what may be referred to as a loss of heterozygosity between a plurality of samples. For example, a first sample may be associated with a normal tissue sample and may have a heterozygous genotype call at a particular sequence position and a second sample may be associated with a tumor tissue from the same individual as the first sample and have a homozygous genotype call at the same position. In the present example, manager 323 may identify the loss of heterozygosity between the two samples. Examples of systems and methods for identifying and representing loss of heterozygosity are presented in U.S. patent application Ser. No.10/219,503, titled “System, Method, and Computer Software for Genotyping Analysis and Identification of Allelic Imbalance”, filed Aug. 15, 2002, incorporated by reference above.

In some embodiments, sequence data manager 323 may then forward the results from data filters 325, genotype call generator 335, and data reliability tester 345, and loss of heterozygosity, for assembly into one or more implementations of sample genotype call data 350 by data assembler 325, as illustrated in step 735. Data 350 may contain the results that correspond to all samples, or alternatively there may be a separate data file 350 that corresponds to each sample. For example, the genotype call results from sample emission intensity data 145′, 145″, and 145′″ may be combined into one sample genotype data 350. In the present example, there could be also separate sample genotype data 350 for each sample emission intensity data 145.

Those of ordinary skill in the related art will appreciate that a number of different genotyping algorithms may be implemented to make genotype calls based, at least in part, upon sample emission intensity data from one or more scanned probe arrays, and that the example algorithm described above is for the purpose of illustration only and should not be limiting in any way.

Output manager 230 may then receive the one or more sets of data 350 from manager 323. In some embodiments output manager 360 may store each of set of data 350 locally in one or more locations such as, for instance, probe array data files 123, or alternatively store each set of data 350 remotely in one or more computers servers, or other means of remote storage. In addition or alternatively the data associated with each set of data 350 may be stored in one or more databases such as, for instance, the Affymetrix® Information Management System (hereafter referred to as AIMS) that could be located locally or remotely.

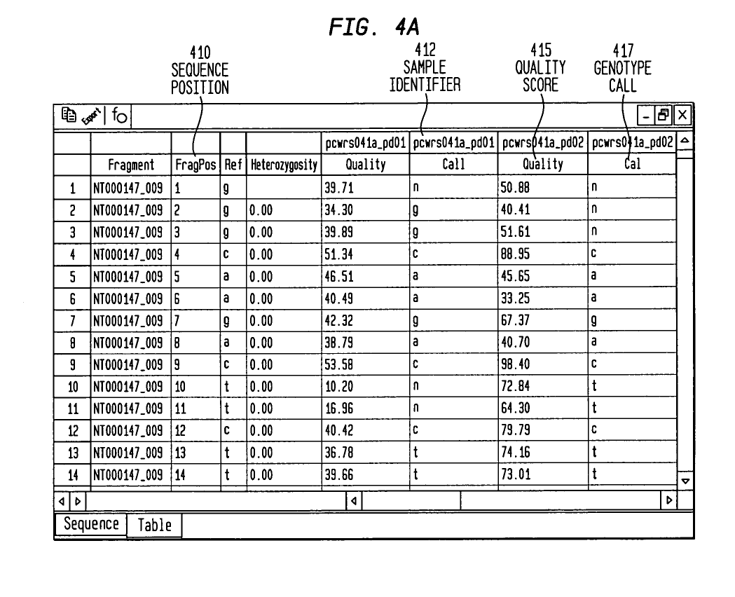

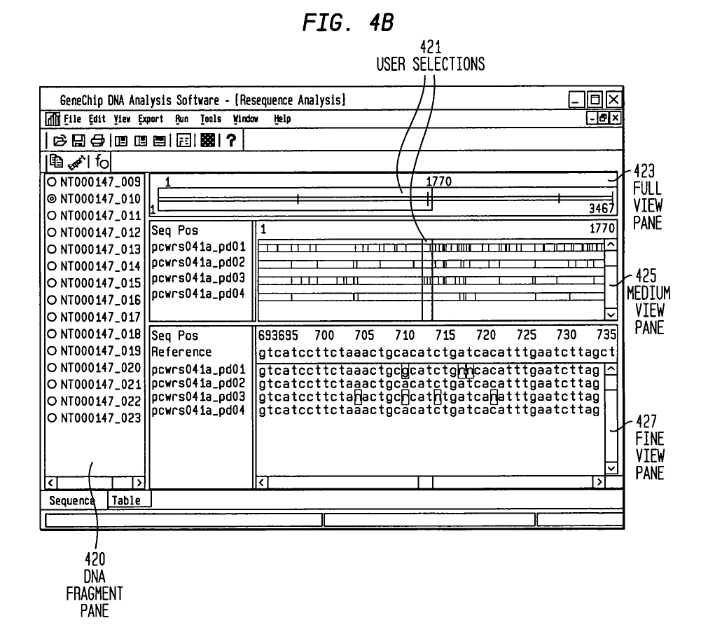

As illustrated in step 740, output manager 230 may arrange the genotype calls from each sample for presentation to the user in one or more graphical user interfaces, hereafter referred to as GUI's. A GUI may be arranged with one or more panes that in turn may each present information in a graphical or tabular format, such as the examples illustrated in FIGS. 4A, 4B, 5, and 6.

FIG. 4A is an illustrative example of a GUI constructed and arranged in a tabular format. In the present example the data is arranged in rows and columns. Some columns include sequence position 410, sample identifier 412, Quality score 415, and genotype call 417. Each row of the present example represents a sequence position and related genotype calls and quality scores for that position. Each row may increment the sequence position by one position such that all positions within a selected sequence may be represented, or alternatively may represent specific non-adjacent positions that could include SNP positions.

FIG. 4B is an illustrative example of a GUI constructed and arranged in a graphical format. In one embodiment, the GUI window may be organized into a plurality of different panes. In the present example, DNA fragment pane 420 may display fragment information that may give the user an indication of the region of DNA sequence being displayed, or regions for which there may be data to display. Full view pane 423 may display the entire length of the sequence that may have been selected by the user that could for instance include a chromosome, contig, plasmid, or other type of sequence that may be associated with a genome. Pane 423 may display the total number of sequence positions in the selected sequence, as well as a feature that may enable a user selection of a portion of the sequence to be displayed in greater detail. For example, user selection 421 may be made by means known to those of ordinary skill in the related art such as, for instance, selection of a sequence range using a mouse to click and drag to define a region within full pane view 423. In the present example, user selection 421 within pane 423 may define the sequence region and associated resolution of that region displayed within medium view pane 425.

As described above in reference to the previous example, user selection 421 in pane 423 may be displayed in medium view pane 425, where the information corresponding to one or more samples each associated with the same sequence region may be aligned for comparison. Pane 425 may color code, or in some other way graphically display sequence positions or regions that may be of interest to the user. Similarly a user may make selection 421 in pane 425 may that enables the display of the selected sequence region associated with selection 421 in pane 425 in greater detail in fine view pane 427.

In response to the user selection, output manager 360 may display the region of sequence associated with selection 421 in pane 425 in pane 427. Pane 427 may display the nucleic acid composition of the sequence that was derived from the genotype calls from manager 323 for each of the corresponding samples. In the example illustrated in FIG. 4B, some sequence positions may be color coded or otherwise graphically distinguished to represent aspects that manager 323 identified. For instance a blue color at a sequence position may mean that the position was assigned as a no call (n), a green color could indicate a heterozygote call, and orange color could indicate a homozygote call. Additionally, manager 323 may indicate identified SNP's in a similar manner. The previous example is used for the purposes of illustration only and should not be limiting in any way. A variety of colors or other graphical representations may be used to indicate a variety of possible features.

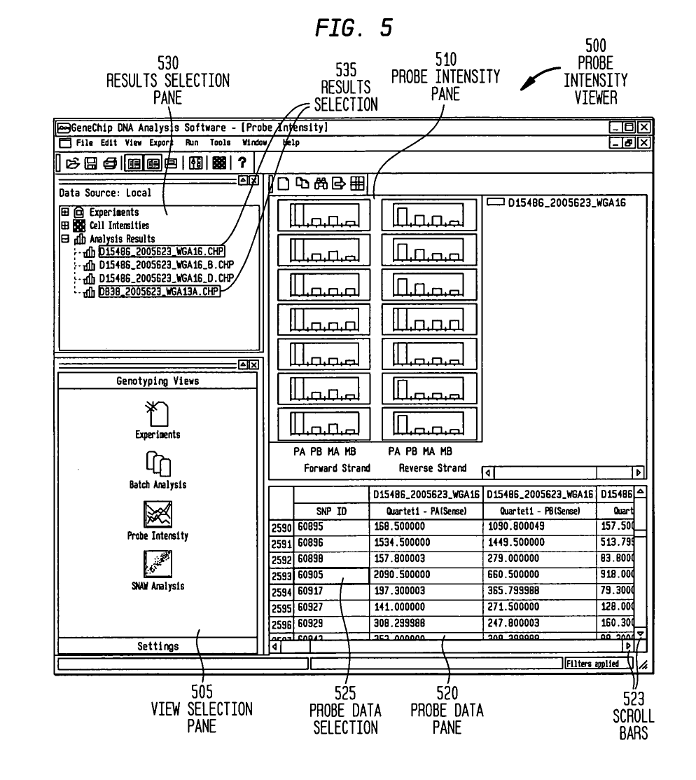

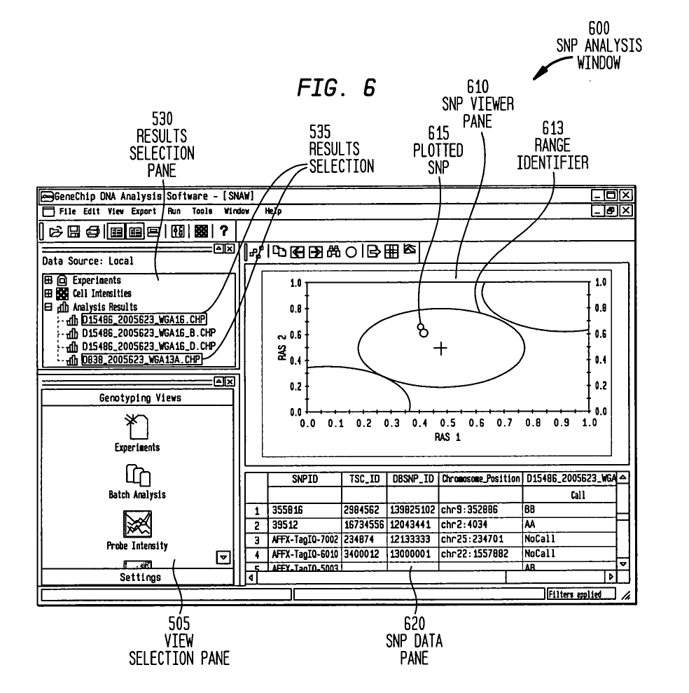

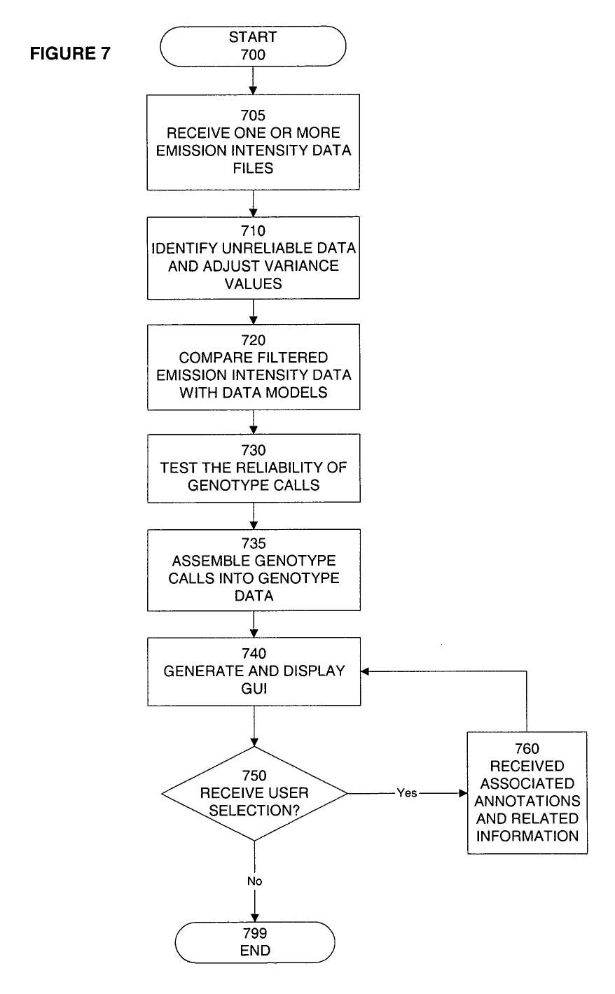

In some embodiments, output manager 360 may generate one or more GUI's such as those illustrated in FIGS. 5 and 6. In the example present in FIG. 5, view selection pane 505 may be displayed to user 175 where user 175 may then make a selection of presentation views from a plurality of options. FIG. 5 further illustrates probe intensity viewer 500 that may represent one such selection in pane 505.

Probe intensity viewer 500 may include a plurality of additional panes such as probe intensity pane 510, probe data pane 520, and results selection pane 530. In some embodiments, user 175 may select one or more sets of results to display simultaneously using methods know to those of ordinary skill in the related art such as, for instance, by placing a cursor over the representation of the desired results using a mouse and clicking a button to complete the selection. An illustrative example of such a selection is presented in FIG. 5 as results selection 535.

Upon entry of results selection 535, output manager 360 may then display graphical and tabular information associated with the selection 535 in one or more panes. For example, probe intensity pane 510 may present a graphical representation depicting the relative detected emission intensities of the probes that belong to a particular probe set. In some implementations it may be desirable to have multiple copies of the same probe set distributed on a probe array that provides redundancy that, for instance, may reduce the probability of certain types of experimental error. In the example of probe intensity pane 510, the detected emission intensities for the probes from multiple probe sets may be graphically displayed as bar graphs, or other type of graphical depiction.

Similarly, the information displayed in probe data pane 520, may be responsive to results selection 535. For example, probe data pane 520 may present information in a tabular format that may include a plurality on rows and columns, where each row may, for instance, be associated with a particular SNP or sequence position and each column may include an identifier, emission intensity value, sequence position, or other type of related information.

Presented in FIG. 6 is SNP analysis window 600 that, similar to probe intensity window 500, may be displayed in response to a selection from view selection pane 505. SNP analysis window may also include a plurality of panes such as results selection pane 530, SNP viewer pane 510 and SNP data pane 520. The functionality of results selection pane 530 associated with SNP analysis window 600 is the same as that described above with respect to probe intensity viewer 500. Similarly, SNP data pane 620 may present information in a tabular format that may include a plurality on rows and columns, where each row may, for instance, be associated with a particular SNP or sequence position and each column may include an identifier, emission intensity value, sequence position, or other type of related information. SNP viewer pane 610 may, for instance, present a graphical representation of the relative quality of the SNP call based, at least in part, upon the calculated RAS value for the coding and non-coding strands. For example, an AB call may be associated with an RAS value of 0.5. If the RAS values from both the coding and non-coding strands are in agreement within a specified range, indicated by range identifier 613, it will be called as AB. In the present example, plotted SNP 615 would have an AB call with an RAS1 value of ˜0.4 and an RAS2 value of ˜0.6. User 175 would be able to make a visual determination of the relative quality of the SNP call based, at least in part on the proximity of plotted SNP 615 to range identifier 613.

In some embodiments, output manger 360 may retrieve information from one or more local or remote sources in response to a selection by user 175 such as, for instance, results selection 535, probe data selection 525, or other type of user selection. For example, output manager may communicate via internet 299 with one or more remote sources such as genomic portal 200. In the present example, genomic portal 200 may include the NetAffx™ web site from Affymetrix®, Inc. of Santa Clara Calif. Output manager 360 may use one or more identifiers such as, for instance, a probe set identifier, SNP identifier, or other type of identifier associated with a user selection to retrieve annotation, sequence, or other type of related information. Output manager 360 may then display the retrieved information in one or more panes of an open GUI window such as SNP analysis window 600 or alternatively open a new GUI window for display. In some implementations, genotype manager 360 may also add retrieved information to sample genotype data 350.

Having described various embodiments and implementations, it should be apparent to those skilled in the relevant art that the foregoing is illustrative only and not limiting, having been presented by way of example only. Many other schemes for distributing functions among the various functional elements of the illustrated embodiment are possible. The functions of any element may be carried out in various ways in alternative embodiments. For example, some or all of the functions described as being carried out by output manager 360 could be carried out by sequence data manager 323, or these functions could otherwise be distributed among other functional elements. Also, the functions of several elements may, in alternative embodiments, be carried out by fewer, or a single, element. For example, the functions of output manager 360 and sequence data manager 323 could be carried out by a single element in other implementations. Similarly, in some embodiments, any functional element may perform fewer, or different, operations than those described with respect to the illustrated embodiment. Also, functional elements shown as distinct for purposes of illustration may be incorporated within other functional elements in a particular implementation.

Also, the sequencing of functions or portions of functions generally may be altered. Certain functional elements, files, data structures, and so on, may be described in the illustrated embodiments as located in system memory of a particular computer. In other embodiments, however, they may be located on, or distributed across, computer systems or other platforms that are co-located and/or remote from each other. For example, any one or more of data files or data structures described as co-located on and “local” to a server or other computer may be located in a computer system or systems remote from the server. In addition, it will be understood by those skilled in the relevant art that control and data flows between and among functional elements and various data structures may vary in many ways from the control and data flows described above or in documents incorporated by reference herein. More particularly, intermediary functional elements may direct control or data flows, and the functions of various elements may be combined, divided, or otherwise rearranged to allow parallel processing or for other reasons. Also, intermediate data structures or files may be used and various described data structures or files may be combined or otherwise arranged. Numerous other embodiments, and modifications thereof, are contemplated as falling within the scope of the present invention as defined by appended claims and equivalents thereto.

What is claimed is:

1. A method for displaying genotype information associated with probe array experiments, comprising the acts of:
receiving one or more sets of emission intensity data, wherein each set of emission intensity data includes a plurality of emission intensity values each associated with a probe disposed upon a probe array;
generating a plurality of genotype calls, wherein each of the genotype calls is based, at least in part, upon one or more of the emission intensity values;
assembling the plurality of genotype calls into one or more genotype data sets; and
displaying each of the one or more genotype data sets in one or more panes of a graphical user interface.

2. The method of claim 1, wherein:
each of the plurality of emission intensity values corresponds to detected emissions from a scanned probe array.

3. The method of claim 1, wherein:
the probe includes a genotyping probe.

4. The method of claim 3, wherein:
the genotyping probe includes a sequencing probe.

5. The method of claim 3, wherein:
the genotyping probe includes a SNP probe.

6. The method of claim 1, wherein:
the genotype call is an A, G, C, T, or (n) call.

7. The method of claim 1, wherein:
the genotype call includes a SNP call.

8. The method of claim 1, wherein:
the one or more panes includes a tabular format

9. The method of claim 1, wherein:
the one or more panes includes a graphical format.

10. The method of claim 8, wherein:
the graphical format includes a representation of relative SNP call quality.

11. The method of claim 8, wherein:
the graphical format includes the plurality of genotype calls associated with a representation of a sequence.

12. The method of claim 8, wherein:
the graphical format includes a representation of probe intensity.

13. The method of claim 1, further comprising the acts of:
retrieving annotation information in response to a user selection of one or more of the displayed genotype calls; and
displaying the annotation information in one or more panes of the graphical user interface.

14. A system for displaying genotype information associated with probe array experiments, comprising:
a sequence data manager constructed and arranged to receive one or more sets of emission intensity data, wherein each set of emission intensity data includes a plurality of emission intensity values each associated with a probe disposed upon a probe array;
a genotype call generator constructed and arranged to generate a plurality of genotype calls, wherein each of the genotype calls is based, at least in part, upon one or more of the emission intensity values;
a data assembler constructed and arranged to assemble the plurality of genotype calls into one or more genotype data sets; and
an output manager constructed and arranged to display each of the one or more genotype data sets in one or more panes of a graphical user interface.

15. The system of claim 14, wherein:
each of the plurality of emission intensity values corresponds to detected emissions from a scanned probe array.

16. The system of claim 14, wherein:
the probe includes a genotyping probe.

17. The system of claim 16, wherein:
the genotyping probe includes a sequencing probe.

18. The system of claim 16, wherein:
the genotyping probe includes a SNP probe.

19. The system of claim 14, wherein:
the genotype call is an A, G, C, T, or (n) call.

20. The system of claim 14, wherein:
the genotype call includes a SNP call.

21. The system of claim 14, wherein:
the one or more panes includes a tabular format

22. The system of claim 14, wherein:
the one or more panes includes a graphical format.

23. The system of claim 22, wherein:
the graphical format includes a representation of relative SNP call quality.

24. The system of claim 22, wherein:
the graphical format includes the plurality of genotype calls associated with a representation of a sequence.

25. The system of claim 22, wherein:
the graphical format includes a representation of probe intensity.

26. The system of claim 14, wherein:
the output manager is further constructed and arranged to retrieve annotation information in response to a user selection of one or more of the displayed genotype calls, and display the annotation information in one or more panes of the graphical user interface.

27. A computer system for displaying genotype information associated with probe array experiments, comprising:
a user computer having system memory with executable code stored thereon, wherein the executable code is constructed and arranged to perform the acts of;
receiving one or more sets of emission intensity data, wherein each set of emission intensity data includes a plurality of emission intensity values each associated with a probe disposed upon a probe array;
generating a plurality of genotype calls, wherein each of the genotype calls is based, at least in part, upon one or more of the emission intensity values;
assembling the plurality of genotype calls into one or more genotype data sets; and
displaying each of the one or more genotype data sets in one or more panes of a graphical user interface.
